(19) United States
(12) Patent Application Publication
(10) Pub. No.: US 2021/0084956 A1
(43) Pub. Date: Mar. 25, 2021

(54) COMPOSITIONS AND METHODS FOR HUNGER CONTROL AND WEIGHT MANAGEMENT

(71) Applicant: AMBRA BIOSCIENCE LLC, San Marcos, CA (US)

(72) Inventors: Kim CHEN, San Marcos, CA (US); Martin BROWN, San Marcos, CA (US); Clegg HUBBELL, San Marcos, CA (US)

(21) Appl. No.: 16/975,045

(22) PCT Filed: Feb. 22, 2019
(86) PCT No.: PCT/US2019/019297

§ 371 Date: Aug. 21, 2020

Related U.S. Application Data
(60) Provisional application No. 62/634,343, filed on Feb. 23, 2018.

(51) Int. Cl. A23L 33/125; A23L 33/00; A23L 33/115; A23L 29/30; A61K 31/7004; A61K 9/28

(52) U.S. Cl. CPC A23L 33/125; A23L 33/30; A61K 9/28; A23L 29/30; A61K 31/7004; A23L 33/115

(57) ABSTRACT

The present disclosure relates to methods and compositions for weight management and/or hunger control combining a bolus dose of a taste receptor agonist targeting enteroendocrine cells beyond the stomach with food products.

### FIELD OF THE INVENTION

The present disclosure relates to methods and compositions for weight management and/or hunger control using taste receptor agonists.

### BACKGROUND OF THE INVENTION

Obesity is an increasing problem throughout the developed world. In the U.S., for example, according to a 2017 study by the Centers for Disease Control nearly 40 percent of adults and about 20 percent of adolescents are obese. Despite the longstanding, massive, effort to develop effective treatments for obesity and uncontrolled appetite, and because of the availability of high calorie foods and an increasing sedentary lifestyle, the number of people overweight worldwide is rapidly growing. One study projects that 45% of the US population and 48% of the British population will be obese by the year 2030. (Wang et al. Health and economic burden of the projected obesity trends in the USA and the UK. Lancet 2011; 378: 815-825.). While genetics may play a role in determining an individuals' likelihood of being overweight, appetite and food intake still play the largest roll in weight gain and obesity. (See Grimm et al. “Genetics of eating behavior: established and emerging concepts.” Nutr Rev 2011; 69: 52-60.)

As of 2017, there are five FDA approved anti-obesity drugs in the U.S., including orlistat (brand name Xenical), lorcaserin (brand name Belviq), phentermine/topiramate (brand name Qsymia), naltrexone/bupropion (brand name Contrave), and liraglutide (brand name Saxenda). The newer drugs are not without potential problems. For example, “the European Committee on Human Medicinal Products has voiced some concerns about the potential long-term risk of cancer schwannoma, astrocytoma, squamous cell carcinoma, breast fibroadenoma, breast adenocarcinoma, and depression in patients taking lorcaserin.” Daneschvar et al. “FDA-Approved Anti-Obesity Drugs in the United States.” Am J Med. 2016 August; 129(8):879.e1-6. In addition, there is concern that the combination drug of phentermine/topiramate induces new onset anxiety and depression and liraglutide should be avoided in patients at risk for hypoglycemia. See id. Surgical treatments including gastric bypass surgery and gastric banding are available, but only in extreme cases. These procedures can be dangerous, and furthermore are not appropriate options for patients with more modest weight loss goals.

Certain intestinal cells, L cells, have been reported to produce GLP-1 in response to glucose, fat and amino acid stimulation. These and other such “enteroendocrine cells” also produce other hormones involved in processes relating to glucose and fuel metabolism, including oxyntomodulin, reported to ameliorate glucose intolerance and suppress appetite, PYY (peptide YY), also observed to suppress appetite, CCK (cholecystokinin), which reportedly stimulates the digestion of fat and protein and also reduces food intake, GLP-2, which reportedly induces gut cell proliferation, and GIP (gastric inhibitory polypeptide, also called glucose-dependent insulinotropic peptide), an incretin secreted from the intestinal K cells that has been observed to augment glucose-dependent insulin secretion. (See, e.g., Jang, et al., 2007, “Gut-expressed gustducin and taste receptors regulate secretion of glucagon-like peptide-1,” Proc Nat Acad Sci 104(38):15069-74 and Parlevliet, et al., 2007, “Oxyntomodulin ameliorates glucose intolerance in mice fed a high-fat diet,” Am J Physiol Endocrinol Metab 294(1):E142-7).

Obesity is associated with a reduced postprandial GLP-1 response. (See e.g., Adam et al. “Glucagon-like peptide-1 release and satiety after a nutrient challenge in normal-weight and obese subjects.” Br J Nutr 2005; 93: 845-851; and Carroll et al. “Influence of BMI and gender on postprandial hormone responses.” Obesity 2007; 15: 2974-2983.). In addition to peripheral locations such as the gut and pancreatic islets, GLP-1 receptors are also found in areas of the brain associated with food intake, appetite, and energy balance. Known roles of GLP-1 include promoting the release of insulin while inhibiting the release of glucagon (See e.g., Willms et al. “Gastric emptying, glucose responses, and insulin secretion after a liquid test meal: effects of exogenous glucagon-like peptide-1 (GLP-1)-(7-36) amide in type 2 (noninsulin-dependent) diabetic patients.” J Clin Endocrinol Metab 1996; 81: 327-332.), and activating the ileal brake to enhance satiety (See Madsbad S. “The role of glucagon-like peptide-1 impairment in obesity and potential therapeutic implications.” Diabetes Obes Metab 2014; 16: 9-21.

PYY is co-secreted with GLP-1 by the intestinal L cells and also plays a role in promoting satiety by way of the ileal brake. (See Pironi et al. “Fat-induced ileal brake in humans: a dose-dependent phenomenon correlated to the plasma levels of peptide YY.” Gastroenterology 1993; 105: 733-739.). Generally, maximal PYY levels are achieved within 120 minutes of eating a meal; however, as with GLP-1, obesity is also associated with a reduced postprandial PYY response. (See e.g., Brownley et al. “Effect of glycemic load on peptide-YY levels in a biracial sample of obese and normal weight women.” Obesity 2010; 18: 1297-1303; Cahill et al. “Serum peptide YY in response to short-term overfeeding in young men.: Am J Clin Nutr 2011; 93: 741-747; and Zwirska-Korczala et al. “Basal and postprandial plasma levels of PYY, ghrelin, cholecystokinin, gastrin and insulin in women with moderate and morbid obesity and metabolic syndrome.” J Physiol Pharmacol 2007; 58: 13-35.

The prior art has previously sought to capitalize on these potentially beneficial hormonal effects by delivering smaller amounts of tastants beyond the stomach, with an emphasis on the delivery of non-nutritive tastant compounds in particular. See, e.g. U.S. Pat. No. 8,828,953. More recently, researchers have advocated solely for colonic delivery of such compounds, thereby avoiding or eliminating any possible nutrient absorption associated with such delivery. See, e.g. Szewczyk et al. “Colonic delivery of nutrients for management of blood glucose in type 2 diabetes patients” Functional Foods In Health and Disease 7(1):36-53 (2017); see also U.S. Pat. Nos. 8,470,885; 8,680,085; 9,006,288; 9,301,938; and 9,314,444. Despite all of these concerted efforts, however, the level of L-cell stimulation and consequent increase in gastrointestinal hormone secretion actually achieved with these various approaches has been only modest, at best. Id.

Accordingly, despite the progress made in the understanding the complex mechanisms controlling appetite, food intake, and energy balance, there is still a lack of effective and readily employable methods and compositions to prevent and/or reduce obesity. Accordingly, there is a clear need in the art to develop safe and effective dietary compositions to assist individuals in controlling their hunger and reducing food intake.

### SUMMARY OF THE INVENTION

The present invention is based, at least in part, on the surprising finding that the targeted delivery of a bolus dose of at least one nutritive taste receptor agonist to enteroendocrine cells acts synergistically with contemporaneously absorbed nutrients in the stomach to dramatically increase the circulating levels of satiety-inducing hormones such as, e.g., GLP-1 and PYY, well above the levels achieved with either agonist or nutrients alone. In certain aspects, therefore, the invention provides dietary compositions and methods for weight management, hunger control and/or appetite suppression, comprising a bolus dose of a nutritive taste receptor agonist formulated for delayed release to enteroendocrine cells combined with a food product for oral ingestion. The food product may be in the form of a bar, cookie, cracker, wafer, or the like comprising a bolus dose of the nutritive taste receptor agonist or, alternatively, the bolus dose of the nutritive taste receptor agonist can be added directly by a subject to any other desired food product, as described in more detail herein. In particular embodiments, the food product is a fixed-calorie and/or low-calorie food product.

Preferably, the taste receptor agonist is nutritive such that it can be metabolized as an energy source, e.g. food or metabolites. In some embodiments, the taste receptor agonist is a sweet receptor agonist, e.g. a sugar. In some embodiments, the sugar is a monosaccharide. In other embodiments the sugar is a disaccharide. In some embodiments, the sugar has a linear structure. In other embodiments the sugar has a ring structure. The sugar may have 3, 4, 5, 6, 7, 8, 9, 10, 11, or 12 carbons. In some embodiments, the sugar is glucose, fructose, or sucrose. In a preferred embodiment, the sugar is glucose.

In some embodiments, the bolus dose comprises at least about 2 grams, at least about 3 grams, at least about 4 grams, at least about 5 grams, at least about 6 grams, at least about 7 grams, at least about 8 grams, at least about 9 grams, at least about 10 grams, at least about 12 grams, or at least about 15 grams of the nutritive taste receptor agonist.

In accordance with the inventive findings provided herein, the taste receptor agonist further includes a delayed-release component (e.g. an enteric and/or timed-release coating) that releases the taste receptor agonist beyond the stomach of a subject, i.e. in the duodenum, the jejunum, the ileum, the caecum, the colon, or combinations thereof. Preferably, the delayed-release component resists degradation in the stomach. In some embodiments, less than about 50%, less than about 25%, less than about 20%, less than about 15%, less than about 10%, less than about 5%, less than about 2.5%, or less than about 1% of the bolus dose of a taste receptor agonist is released in the stomach of said subject.

In one aspect, the invention is directed to an edible food product. In some embodiments, the food product is provided as a single serving (such as a single bar, cookie, cracker, wafer, or the like) comprising a bolus dose of the taste receptor agonist-containing additive. In alternative embodiments, a pre-measured bolus dose of the taste receptor agonist-containing additive is provided to the consumer in a vial, cylinder, sachet or other suitable container for combination with any other desired food product (such as a shake, yogurt, custard, pudding, frozen food or other meal preparation).

In specific embodiments, the additive comprises microencapsulated taste receptor agonist particles. Such particles can have a range of characteristics. For example, the particles can be formed using a specific coating material, such as a coating material that is safe for human consumption. In one embodiment, the coating material comprises a hydrogenated fat. The hydrogenated fat may be a fully-hydrogenated fat. In other embodiments, the particles can have a specific content of taste receptor agonist. For example, the particles can comprise about 10% to about 90% by weight taste receptor agonist. In some embodiments, the particles comprise about 40% to about 60% by weight taste receptor agonist. The microencapsulated taste receptor agonist particles can also be of a particular size, such as being of a size of less than about 0.7 mm. The single serving of the food product preferably includes the taste receptor agonist in a specific amount. For example, in one embodiment, the additive is present in a content sufficient so that the single serving of the food product includes about 1 gram to about 50 grams of taste receptor agonist.

In some embodiments, the delayed-release component comprises an enteric coating, a timed-release coating, or a combination thereof. In some embodiments, the enteric coating has an onset of release at about pH 5.0 or above, about pH 5.5 or above, about pH 6.0 or above, about pH 6.5 or above, about pH 7.0 or above, or combination thereof following ingestion by a subject. In some embodiments, the timed release coating releases less than 30, 40, 50, or 60% of said taste receptor agonist after 1, 1.5, or 2 hours in 0.1 N HCl.

In preferred embodiments, the delayed-release component of the bolus dose comprises an additional taste receptor agonist, e.g., a fat receptor agonist, which can function as a timed-release system. Non-limiting examples of fat receptor agonists include linoleic acids, oleic acids, palmitates, oleoylethanolamides, omega-3 fatty acids, mixed fatty acid emulsion, and N-acylphosphatidylethanolamine (NAPE), myristoleic acid, palmitoleic acid, alpha-linolinic acid, arachidonic acid, eicosapentaenoic acid, erucic acid, docosahexaenoic acid. Particularly preferred are Generally-Recognized As Safe (i.e. GRAS) compounds such as, e.g., fully hydrogenated fats such as soybean oil and the like.

In the exemplary embodiments detailed herein, the coating comprises a fully hydrogenated fat, e.g. fully hydrogenated soybean oil. In some embodiments, the delayed-release component may comprise at least about 40, 45, or 50% (w/w) of the overall bolus dose, and preferably at least about 55, 60 or 65% (w/w) of the overall bolus dose.

In some embodiments, the food product comprises a carbohydrate, a fat, a protein, as well as their components such as sugars, fatty acids and amino acids, or a combination thereof. In some embodiments, the food product comprises up to about 10%, up to about 20%, up to about 30%, up to about 40%, up to about 50%, up to about 60%, up to about 70%, up to about 80%, up to about 90%, or up to about 100% carbohydrate and/or sugar. In some embodiments, the food product comprises up to about 10%, up to about 20%, up to about 30%, up to about 40%, up to about 50%, up to about 60%, up to about 70%, up to about 80%, up to about 90%, or up to about 100% fat and or fatty acids. In some embodiments, the food product comprises up to about 10%, up to about 20%, up to about 30%, up to about 40%, up to about 50%, up to about 60%, up to about 70%, up to about 80%, up to about 90%, or up to about 100% protein and/or amino acids.

In some embodiments, the food product comprises at least about 100 calories, at least about 150 calories, at least about 200 calories, at least about 250 calories, or at least about 300 calories. In an alternative aspect, the nutrient component comprises less than about 500, less than about 450, less than about 400, or less than about 350 calories.

In another aspect, the invention provides satiety-inducing food products, and preferably fixed-calorie and/or low-calories food products, comprising a plurality of microencapsulated particles comprising at least one nutritive taste receptor agonist formulated for delayed release to enteroendocrine cells, wherein said particles collectively comprise at least about 2 grams of said at least one taste receptor agonist, and said food product comprises at least about 50, 75, 100, 125, 150, 175, 200, 225 or 250 calories.

In additional aspects, the invention includes a method of inducing satiety, controlling hunger, and/or suppressing appetite in a subject comprising administering to said subject a dietary composition comprising a bolus dose of a nutritive taste receptor agonist formulated for delayed release to enteroendocrine cells in combination with an edible food product, and preferably a fixed- and/or low-calorie food product. In some embodiments, the food product comprises the bolus dose of nutritive taste receptor agonist when provided to the subject. In some embodiments, a pre-measured bolus dose of the nutritive taste receptor agonist in the form of, e.g., particles, is provided to the subject in a vial, cylinder, sachet or other suitable container for direct addition by the subject to any desired food product, e.g. a shake, yogurt, custard, pudding, and/or frozen or other meal preparation.

In some embodiments, administration of the dietary composition increases the circulating concentration of GLP-1 (total), GLP-1 (active), GLP-2, PYY (total), PYY 3-36, or combinations thereof. In some embodiments, the circulating concentrations of GLP-1 (active) and/or PYY (total) are increased by at least about 10%, by at least about 15%, by at least about 20%, by at least about 25%, by at least about 50%, by at least about 75%, by at least about 100%, by at least about 150%, or by at least about 200% compared to placebo-controlled circulating concentrations. The subject may be a human or a domesticated animal. Preferably, the subject is a human.

In another aspect, kits are provided comprising a bolus dose of microencapsulated particles comprising at least one nutritive taste receptor agonist formulated for delayed release to enteroendocrine cells, wherein said particles collectively comprise at least about 2 grams of said at least one taste receptor agonist, provided in a vial, cylinder, sachet or other suitable container together with instructions for use in combination with food products. In some embodiments, the at least one nutritive taste receptor agonist is a sweet receptor agonist. Preferably the sweet receptor agonist is a sugar a selected from the group consisting of glucose, fructose, or sucrose. In particular embodiments, the at least one taste receptor agonist is formulated with a timed-release coating comprising a hydrogenated fat, preferably a fully-hydrogenated fat. In exemplary embodiments, the hydrogenated fat comprises at least about 50% (w/w) of said bolus dose.

In yet another aspect, invention further comprises ingredients well known in the art such as preservatives, flavor enhancers, wetting agents, and emulsifiers.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The present disclosure is disclosed with reference to the accompanying drawings, wherein:

FIG. 1: Tastant and nutrient synergy. The effects of enteric coated tastants (Active) versus placebo (PBO) and nutrients (75 g glucose) on circulating gut hormone concentrations were evaluated in a 12 subject, randomized, double blind, placebo-controlled, crossover study that assessed the effects of tastants test article LC001 and nutrients on the circulating gut satiety hormone (PYY). Subjects were administered the study test article in the morning after an overnight fast at t=0 min, and were given a 75 g glucose challenge at t=60 min, unless they were being tested for the fasting condition. Blood was sampled between t=−30 and 300 min relative to the dosing of test article for determination of gut hormone (PYY total) concentrations as depicted in FIG. 1A. Results are shown in FIG. 1B with the average baseline subtracted circulating PYY-total (pg/mL) plotted on the y-axis against time (in minutes) on the x-axis.

FIG. 2: Efficacy of tastant and nutrient synergy bars for use in intermittent calorie restriction diet for weight loss. Ten individuals (6 females, 4 males, average age 58.4 years) were instructed and trained to limit their total daily calorie intake on 3 non-consecutive days per week to no more than 500 calories, and to not limit calorie consumption on the other 4 days with and without using nutrition bars specially formulated as described herein. Body weights were measured at the beginning of and after 7 days of intermittent calorie restriction dieting and the results are illustrated in FIG. 2.

FIG. 3: Effect of oil:core ratio on release of encapsulated material. Duplicate samples (about 30 mg each) were transferred to 20-mL vials. 10 mL of 0.1N HCl (preheated to 37° C.) was added to the vials. The vials were placed in 37° C. oven on shaker table set for gentle mixing. Aliquots were removed after 10 minutes, and 1 and 2 hours. These aliquots were filtered (0.45 μm) and assayed for Core ingredient content by HPLC.

### DETAILED DESCRIPTION

The present invention contemplates the surprising finding that the targeted delivery of a bolus dose of nutritive taste receptor agonist(s) to enteroendocrine cells acts synergistically with contemporaneously absorbed nutrients in the stomach to dramatically increase the circulating levels of satiety-inducing hormones such as, e.g., GLP-1 and PYY, well above the levels achieved with either agonist or nutrients alone. In one aspect, therefore, the invention provides dietary compositions and methods for weight management, hunger control and/or appetite suppression, comprising a bolus dose of a nutritive taste receptor agonist formulated for delayed release to enteroendocrine cells combined with a food product for oral ingestion. The food product may be in the form of a bar, cookie, cracker, wafer, or the like comprising a bolus dose of the nutritive taste receptor agonist or, alternatively, a bolus dose of the nutritive taste receptor agonist can be added directly by a subject to any other desired food product, as described in more detail herein. In particular embodiments, the food product is a fixed-calorie and/or low-calorie food product.

In some embodiments, one or more nutritive taste receptor agonist(s) are used to modulate the secretion of hormone molecules and regulate metabolic processes including hunger, appetite and/or satiety. In certain embodiments, a nutritive taste receptor agonist(s) is combined with a food product. It is contemplated that the addition of nutritive taste receptor agonist(s) along with a food product may result in enhanced stimulation of hormone release by enteroendocrine cells.

The present embodiments described herein additionally contemplate targeting administration of one or more nutritive taste receptor agonist(s) to specific sites throughout the gut. Enteroendocrine cells, e.g., L cells, K cells, and I cells, that each secrete a different set of metabolic hormones in response to nutritive taste receptor agonist stimulation, occur throughout the length of the intestine. The concentrations and proportions of these enteroendocrine cell types are different in the various intestinal segments, and each cell type has a different metabolic hormone expression profile. Targeted administration of the compositions of the invention to specific intestinal segments, for example, through the use of formulations designed for release within one or more desired segments of the intestine, provides an additional level of control over the effect of such compositions, e.g., in the modulation of hormones involved in metabolism, including satiety.

The present embodiments described herein thus include a novel approach to treating obesity, reducing bodyweight, and/or suppressing and/or controlling hunger and/or appetite and/or satiety by, for example, modulating the secretion of metabolic hormones through enteroendocrine activation via a nutritive taste receptor agonist.

The embodiments described herein include compositions and methods for modulating the concentrations of circulating enteroendocrine cell hormones, including, but not limited to, GLP-1, GLP-2, GIP, oxyntomodulin, PYY, CCK, glycentin, insulin, glucagon, C-peptide, ghrelin, amylin, uroguanylin, etc., such compositions and methods comprising administering at least one nutritive taste receptor agonist formulated for delayed release to enteroendocrine cells combined with and preferably within a food product for oral ingestion.

In some embodiments, two nutritive taste receptor agonists are administered to a subject. In certain embodiments, three nutritive taste receptor agonists are administered to a subject. In yet other embodiments, four nutritive taste receptor agonists are administered to a subject. In yet other embodiments, five nutritive taste receptor agonists are administered to a subject. In further embodiments, six or more nutritive taste receptor agonists are administered to a subject.

Gut hormones secreted by enteroendocrine cells are released from their basolateral aspect into the mesenteric venous circulation. Therefore, these hormones traverse the portal vein area which drains all mesenteric venous efflux. Gut hormones, typically peptides, are often also neurotransmitters and as such can stimulate afferent nerve endings that emanate from the gut and the liver. It is well recognized that CCK causes afferent vagal activation and that its physiologic effects are due almost exclusively to this neural activation. Hormones such as GLP-1, oxyntomodulin, PYY and GIP, and their post DPP-IV degradation breakdown products can have physiologic effects at the level of gut nerves and can activate portal receptor/signaling pathways to cause activation of hepatic afferents. The action of GLP-1 to cause glucose-dependent insulin secretion is thought to predominantly occur via neural activation as its degradation by DPP-IV upon release begins immediately causing its circulating half-life to be less than 2 minutes. Moreover, the portal:arterial gradient for GLP-1 is large (>2:1) thus making its endocrine function in the beta cell excessively inefficient. Given its portal to peripheral gradient and its action as a neurotransmitter to activate gut afferent nerves, and its role to cause portal activation of hepatic afferents it is plausible that GLP-1's physiologic and pharmacologic actions can be produced in the absence of large fluctuations (and even perhaps undetectable alterations) of circulating peripheral (arterial or post hepatic venous) concentrations of GLP-1. As such GLP-1 is akin to norepinephrine which is a neurotransmitter but spills over into the circulation; like GLP-1, norepinephrine can be infused peripherally to act as a hormone to reproduce many of its physiologic functions. Thus, in some embodiments, the compositions and methods provided herein produce salutary effects on blood glucose and weight loss by enhancing portal concentrations of gut hormones while minimally augmenting peripheral concentrations.

Preferably, the taste receptor agonist is nutritive such that it can be metabolized as an energy source, e.g. food or metabolites. In some embodiments, the taste receptor agonist is a sweet receptor agonist, e.g. a sugar. In some embodiments, the sugar is a monosaccharide. In other embodiments the sugar is a disaccharide. In some embodiments, the sugar has a linear structure. In other embodiments the sugar has a ring structure. The sugar may have 3, 4, 5, 6, 7, 8, 9, 10, 11, or 12 carbons. In some embodiments, the sugar is glucose, fructose, or sucrose. In a preferred embodiment, the sugar is glucose.

In some embodiments, the bolus dose described herein comprises at least about 2 grams, at least about 3 grams, at least about 4 grams, at least about 5 grams, at least about 6 grams, at least about 7 grams, at least about 8 grams, at least about 9 grams, at least about 10 grams, at least about 12 grams, or at least about 15 grams of the nutritive taste receptor agonist.

In accordance with the subject invention, the nutritive taste receptor agonist further includes a delayed-release component (e.g. an enteric and/or timed-release coating) that releases the taste receptor agonist beyond the stomach of a subject, i.e. in the duodenum, the jejunum, the ileum, the caecum, the colon, or combinations thereof. Preferably, the delayed-release component resists degradation in the stomach. In some embodiments, less than about 50%, less than about 25%, less than about 20%, less than about 15%, less than about 10%, less than about 5%, less than about 2.5%, or less than about 1% of the bolus dose of a taste receptor agonist is released in the stomach of said subject.

In further embodiments, it is beneficial for the taste receptor agonist to be incorporated directly into the food product in the form of a pre-formed component that is coated or microencapsulated. Coating or microencapsulation can be useful to delay release of the taste receptor agonist.

Any type of coating or encapsulating material useful in preparing components for use in food products can be used according to the invention. Accordingly, the coating material can comprise any material that is a food grade material or is otherwise generally safe for human consumption. Suitable coatings include mono- and di-glycerides, as well as polymeric materials, such as ethylcellulose (EC), methylcellulose, hydroxypropylcellulose (HPC), hydroxypropylmethylcellulose (HPMC), lipids and oils (such as vegetable, seed, or nut oils) and the like. Commercial coating products sold under the names EUDRAGIT™ and DESCOTE™ are specific examples of coatings and encapsulations useful according to the invention.

In other embodiments, the coating or encapsulating material is a fat. The fat may be hydrogenated fat. The fat may be a at least 50%, at least 60%, at least 70%, at least 80%, at least 90%, at least 95%, or 100% hydrogenated. In an exemplary embodiment, fully hydrogenated soybean oil is used.

In specific embodiments, the coating material used to prepare microencapsulated taste receptor agonist comprises a compound that is known under the designation “Generally Recognized As Safe,” or GRAS. The designation GRAS was established by the U.S. Food and Drug Administration (FDA) to encompass chemicals that are safe to be added to foods. GRAS compounds are exempted from the usual Federal Food Drug and Cosmetic Act (FFDCA) food additive tolerance requirements. Accordingly, any material that is on the GRAS list and that could used as a coating material in a microencapsulation process is contemplated in the microencapsulated taste receptor agonist that is incorporated into the food products of the present invention.

Microencapsulation is generally recognized as a process by which small particles or droplets of a material are surrounded by a coating to produce capsules known as microcapsules, which can actually include capsules having sizes in the nanometer to millimeter range. The material inside the capsule is referred to as the core, internal phase or fill, whereas the wall is sometimes called a shell, coating, or membrane.

In certain embodiments, microencapsulated taste receptor agonists according to the invention could be formed using any of various chemical encapsulation techniques such as solvent evaporation, solvent extraction, organic phase separation, interfacial polymerization, simple and complex coacervation, in-situ polymerization, liposome encapsulation, and nanoencapsulation. Alternatively, physical methods of encapsulation could be used, such as spray coating, pan coating, fluid bed coating, annular jet coating, spinning disk atomization, spray cooling, spray drying, spray chilling, stationary nozzle coextrusion, centrifugal head coextrusion, or submerged nozzle coextrusion.

Particle size is preferably adjustable such that a product prepared therewith can exhibit a mildly grainy effect (which may be desirable in some applications) to practically no noticeability of the presence of the microparticles. In specific embodiments, the microencapsulated taste receptor agonist is of a sufficient small particle size such that the presence of the microcapsules is not recognizable to the average human consumer.

Typically, the microparticles are sized to be generally less than about 1 mm in size, preferably less than about 0.8 mm, less than about 0.7 mm, less than about 0.6 mm, less than about 0.5 mm, less than about 0.4 mm, less than about 0.3 mm, less than about 0.2 mm, or less than about 0.1 mm. In further embodiments, the microparticles can be less than about 50 μm, less than about 40 μm, less than about 30 μm, less than about 20 μm, less than about 10 μm, or less than about 1 μm. In certain embodiments, the microparticles have sizes in the range of about 0.2 mm to about 2 mm, about 0.3 mm to about 1.5 mm, about 0.4 mm to about 1 mm, or about 0.5 mm to about 1 mm. In a specific embodiment, the microparticles are sized such that at least 98% of the microparticles are less than 0.6 mm in size. In other embodiments, the microparticles are sized such that less than or equal to 2% of the microparticles are retained on a 30 mesh screen. In still further embodiments, microparticles are of a size making the particles essentially or completely undetectable by a consumer of a product incorporating such particles. In such embodiments, the microparticles can have average sizes in the range of about 1 μm to about 0.8 mm, about 10 μm to about 0.7 mm, about 0.1 mm to about 0.7 mm, about 0.2 mm to about 0.7 mm, or about 0.3 mm to about 0.6 mm. In specific embodiments, the microparticles have an average size of 0.4 mm to about 0.8 mm or about 0.6 mm.

Microparticles can vary in relation to the overall content of the encapsulated material. Microparticles used according to the present invention may comprise predominately the encapsulated material; however, lesser contents are also contemplated. In certain embodiments, the microparticles comprise about 10% by weight taste receptor agonist, based on the overall weight of the microparticles, about 20% by weight taste receptor agonist, about 30% by weight taste receptor agonist, about 40% by weight taste receptor agonist, about 50% by weight taste receptor agonist, about 55% by weight taste receptor agonist, about 60% by weight taste receptor agonist, about 65% by weight taste receptor agonist, about 70% by weight taste receptor agonist, about 80% by weight taste receptor agonist, or about 90% by weight taste receptor agonist based on the overall weight of the microparticles. In a specific embodiment, the microparticles comprise about 10% to about 90% by weight taste receptor agonist, about 20% to about 80% by weight taste receptor agonist, about 30% to about 70% by weight taste receptor agonist, about 30% to about 60% by weight taste receptor agonist, or about 40% to about 60% by weight taste receptor agonist.

In some embodiments, microparticles used according to the present invention may comprise about 40% by weight taste receptor agonist and about 60% by weight a hydrogenated fat. Alternatively, microparticles used according to the present invention may comprise about 50% by weight taste receptor agonist and about 50% by weight a hydrogenated fat, or about 60% by weight taste receptor agonist and about 40% by weight a hydrogenated fat. In these embodiments, the hydrogenated fat may be at least 90%, at least 95%, or 100% hydrogenated.

In certain embodiments, the food products of the invention include from about 1 gram to about 50 grams of taste receptor agonist, about 2 grams to about 40 grams of taste receptor agonist, about 3 grams to about 30 grams of taste receptor agonist, about 5 grams to about 25 grams taste receptor agonist, about 10 grams to about 20 grams taste receptor agonist, about 5 grams to about 15 grams taste receptor agonist, about 10 grams to about 15 grams of taste receptor agonist, or about 25 grams to about 50 grams of taste receptor agonist per serving.

In certain embodiments, the microencapsulated taste receptor agonist used in the present invention consists essentially of the taste receptor agonist and a delayed-release coating material, wherein the coating material is preferably an enteric coating, a timed-release coating, or a combination thereof.

In one embodiment, the release mechanism is a “timed” or temporal release (“TR”) system that releases the taste receptor agonist at certain timepoints subsequent to administration. Timed release systems are well known in the art and suitable timed release system can include any known excipient and/or coating. For example, excipients in a matrix, layer or coating can delay release of an active agent by slowing diffusion of the active agent into an environment. Suitable timed release excipients, include but are not limited to, acacia (gum arabic), agar, aluminum magnesium silicate, alginates (sodium alginate), sodium stearate, bladderwrack, bentonite, carbomer, carrageenan, Carbopol, cellulose, microcrystalline cellulose, ceratonia, chondrus, dextrose, furcellaran, gelatin, Ghatti gum, guar gum, galactomannan, hectorite, lactose, sucrose, maltodextrin, mannitol, sorbitol, honey, maize starch, wheat starch, rice starch, potato starch, gelatin, sterculia gum, xanthum gum, Glyceryl behenate (e.g., Compritol 888 ato), Gylceryl distearate (e.g. Precirol ato 5), polyethylene glycol (e.g., PEG 200-4500), polyethylene oxide, adipic acid, gum tragacanth, ethyl cellulose (e.g., ethyl cellulose 100), ethylhydroxyethyl cellulose, ethylmethyl cellulose, methyl cellulose, hydroxyethyl cellulose, hydroxyethylmethyl cellulose (e.g., K100LV, K4M, K15M), hydroxypropyl cellulose, poly(hydroxyethyl methacrylate), cellulose acetate (e.g. cellulose acetate CA-398-10 NF), cellulose acetate phthalate, cellulose acetate propionate, cellulose acetate butyrate, hydroxypropyl methyl cellulose acetate succinate, hydroxypropyl methyl cellulose phthalate, cellulose butyrate, cellulose nitrate, oxypolygelatin, pectin, polygeline, povidone, propylene carbonate, polyandrides, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl cellulose, sodium carboxymethyl-cellulose (CMC), silicon dioxide, vinyl polymers, e.g. polyvinyl pyrrolidones (PVP: povidone), polyvinyl acetates, or polyvinyl acetate phthalates and mixtures, Kollidon SR, acryl derivatives (e.g. polyacrylates, e.g. cross-linked polyacrylates, methycrylic acid copolymers), Splenda® (dextrose, maltodextrin and sucralose) or combinations thereof. The timed release excipient may be in a matrix with active agent, in another compartment or layer of the formulation, as part of the coating, or any combination thereof. Varying amounts of one or more timed release excipients may be used to achieve a designated release time.

In some embodiments, the timed release systems are formulated to release the taste receptor agonist at an onset of about 5 minutes, about 10 minutes, about 20 minutes, about 30 minutes, about 40 minutes, about 50 minutes, about 60 minutes, about 70 minutes, about 80 minutes, about 90 minutes, about 100 minutes, about 110 minutes, about 120 minutes, about 130 minutes, about 140 minutes, about 150 minutes, about 160 minutes, about 170 minutes, about 180 minutes, about 190 minutes, about 200 minutes, about 210 minutes, about 220 minutes, about 230 minutes, about 240 minutes, about 250 minutes, about 260 minutes, about 270 minutes, about 280 minutes, about 290 minutes, about 300 minutes, about 310 minutes, about 320 minutes, about 330 minutes, about 340 minutes, about 350 minutes, about 360 minutes, about 370 minutes, about 380 minutes, about 390 minutes, about 400, about 400, about 410, or about 420 minutes subsequent to ingestion. In embodiments with multiple releases, timed release systems are formulated to release at more than one time point. In certain embodiments, the timed release systems are formulated to release at an onset of about 10 minutes, about 30 minutes, about 120 minutes, about 180 minutes and about 240 minutes after administration. In certain embodiments of the timed release systems are formulated to release at an onset of about 5 to about 45 minutes, about 105 to about 135 minutes, about 165 to about 195 minutes, about 225 to about 255 minutes or a combination of times thereof following ingestion by a subject.

In some embodiments, the nutritive taste receptor agonist is formulated to release at a duration of about 30 minutes, about 40 minutes, about 50 minutes, about 60 minutes, about 70 minutes, about 80 minutes, about 90 minutes, about 100 minutes, about 110 minutes, about 120 minutes, about 130 minutes, about 140 minutes, about 150 minutes, about 160 minutes, about 170 minutes, about 180 minutes, about 190 minutes, about 200 minutes, about 210 minutes, about 220 minutes, about 230 minutes, about 240 minutes, about 250 minutes, about 260 minutes, about 270 minutes, about 280 minutes, about 290 minutes, about 300 minutes, about 310 minutes, about 320 minutes, about 330 minutes, about 340 minutes, about 350 minutes, about 360 minutes, about 370 minutes, about 380 minutes, about 390 minutes, about 400, about 400, about 410, or about 420 minutes subsequent to the onset of the release described above. In embodiments, the food product comprises nutritive taste receptor agonists with multiple releases with more than one durations of time to completely release.

The nutritive taste receptor agonist may also be coated with an enteric coating, which protects against degradation in an acidic environment, such as the stomach, and allows a delayed release into a target area.

The enteric coating may be, as a non-limiting example, wax or wax like substance, such as carnauba wax, fatty alcohols, hydrogenated vegetable oils, zein, shellac, sucrose, Arabic gum, gelatin, dextrin, psyllium husk powder, polymethacrylates, anionic polymethacrylates, mixtures of poly(methacrylic acid, methyl methacrylate), polymers or copolymers derived from acrylic and/or methacrylic acid esters, cellulose acetate phthalate, cellulose acetate trimelliate, hydroxypropyl methylcellulose phthalate (HPMCP), cellulose propionate phthalate, cellulose acetate maleate, polyvinyl alcohol phthalate, hydroxypropyl methylcellulose acetate succinate (HPMCAS), hydroxypropyl methylcellulose hexahydrophthalate, polyvinyl acetate phthalate, mixtures of poly(methacrylic acid, ethyl acrylate), ethylcellulose, methylcellulose, propylcellulose, chitosan succinate, chitosan succinate, polyvinyl acetate phthalate (PVAP), polyvinyl acetate polymers carboxymethylethyl cellulose and compatible mixtures thereof. In addition, an inactive intermediate film may be provided between the active agent and the enteric coating to prevent interaction of the active agent with the enteric coating.

The enteric coatings can be formulated to release the nutritive taste receptor agonist at a desired pH using combinations of enteric polymers. It is well-known that different locations of the gastrointestinal system have specific pHs. For example, the duodenum may correspond to a pH 5.5 environment and the jejunum may correspond to pH 6.0 environment. In some embodiments, the enteric coatings are formulated to release nutritive taste receptor agonist at an onset of a pH including about pH 1 or above, about pH 1.5 or above, about pH 2 or above, about pH 2.5 or above, about pH 3 or above, about pH 3.5 or above, about pH 4 or above, about pH 4.5 or above, about pH 5 or above, about pH 5.5 or above, about pH 6 or above, about pH 6.5 or above, or about pH 7 or above. In embodiments with multiple releases, the enteric coatings are formulated to release at an onset of two or more pH values. In certain embodiments, the enteric coatings are formulated to release at an onset of pH 5.5, 6.0, 6.5, 7.0, and/or above. In certain embodiments, the enteric coatings are formulated to release at an onset of pH 5.5, 6.0 and 6.5. In certain embodiments, the enteric coatings are formulated to release at the duodenum, jejunum, ileum, and lower intestine. In yet other embodiments, the enteric coatings are used in combination with other release systems such as a timed release system.

In some embodiments, the enteric coating has an onset of release at about pH 5.0 or above, about pH 5.5 or above, about pH 6.0 or above, about pH 6.5 or above, about pH 7.0 or above, or combination thereof following ingestion by a subject. In some embodiments, the timed release coating releases less than 30, 40, 50, or 60% of said taste receptor agonist after 1, 1.5, or 2 hours in 0.1 N HCL.

In preferred embodiments, the subject dietary compositions comprise an additional taste receptor agonist, e.g., a fat receptor agonist, which functions as the timed-release coating system. Non-limiting examples of fat receptor agonists include linoleic acids, oleic acids, palmitates, oleoylethanolamides, omega-3 fatty acids, mixed fatty acid emulsion, and N-acylphosphatidylethanolamine (NAPE), myristoleic acid, palmitoleic acid, alpha-linolinic acid, arachidonic acid, eicosapentaenoic acid, erucic acid, and docosahexaenoic acid.

In the exemplary embodiments detailed herein, the coating comprises a fully hydrogenated fat, e.g., fully hydrogenated soybean oil. In some embodiments, this delayed-release component may comprise at least about 40, 45, or 50% (w/w) of the overall bolus dose, and preferably at least about 55, 60 or 65% (w/w) of the overall bolus dose.

In some embodiments, the food product comprises a carbohydrate, a fat, a protein, as well as their components such as sugars, fatty acids and amino acids, or a combination thereof. In some embodiments, the food product comprises up to about 10%, up to about 20%, up to about 30%, up to about 40%, up to about 50%, up to about 60%, up to about 70%, up to about 80%, up to about 90%, or up to about 100% carbohydrate and/or sugar. In some embodiments, the food product comprises up to about 10%, up to about 20%, up to about 30%, up to about 40%, up to about 50%, up to about 60%, up to about 70%, up to about 80%, up to about 90%, or up to about 100% fat and or fatty acids. In some embodiments, the food product comprises up to about 10%, up to about 20%, up to about 30%, up to about 40%, up to about 50%, up to about 60%, up to about 70%, up to about 80%, up to about 90%, or up to about 100% protein and/or amino acids.

In some embodiments, the food product comprises at least about 50 calories, at least about 100 calories, at least about 150 calories, at least about 200 calories, at least about 250 calories, or at least about 300 calories. In an alternative aspect, the nutrient component comprises less than about 500, less than about 450, less than about 400, or less than about 350 calories.

In another aspect, the invention provides hunger control and/or satiety-inducing food products, and preferably fixed-calorie and/or low-calories food products, comprising a plurality of microencapsulated particles comprising at least one nutritive taste receptor agonist formulated for delayed release to enteroendocrine cells, wherein said particles collectively comprise at least about 2 grams of said at least one taste receptor agonist, and said food product comprises at least about 50, 75, 100, 125, 150, 175, 200, 225 or 250 calories.

In another aspect, the invention includes a method of controlling hunger, suppressing appetite and/or inducing satiety in a subject comprising administering to said subject a dietary composition comprising a bolus dose of a nutritive taste receptor agonist formulated for delayed release to enteroendocrine cells combined with a fixed- and/or low-calorie food product. In some embodiments, administration of the dietary composition increases the circulating concentration of GLP-1 (total), GLP-1 (active), GLP-2, PYY (total), PYY 3-36, or combinations thereof. In some embodiments, the circulating concentrations of GLP-1 (active) and/or PYY (total) are increased by at least about 10%, by at least about 15%, by at least about 20%, by at least about 25%, by at least about 50%, by at least about 75%, by at least about 100%, by at least about 150%, or by at least about 200% compared to placebo-controlled circulating concentrations. The subject may be a human or a domesticated animal. Preferably, the subject is a human.

In yet another aspect, invention further comprises ingredients well known in the art such as preservatives, flavor enhancers, wetting agents, and emulsifiers.

In one aspect, the microencapsulated particles, food products and/or kits further comprise vitamins, minerals, herbs, spices, oils, weight-loss supplements, energy-promoting supplements, and various further dietary supplements that may be useful for providing a particular health benefit or perceived benefit.

A “subject’ may include any mammal, including humans. A “subject” may also include other mammals kept as pets or livestock (e.g., dogs, cats, horses, cows, sheep, pigs, goats). Subjects who may benefit from the methods provided herein may be overweight or obese; however, they may also be lean. Subjects who may benefit from the methods provided herein may be desirous of losing weight or may have an eating disorder, such as binge eating, or an eating condition, such as food cravings. Subjects who may benefit from the methods provided herein may be desirous of reducing caloric intake. Subjects can be of any age. Accordingly, these disorders can be found in young adults and adults (e.g., those aged 65 or under) as well as infants, children, adolescents, and the elderly (e.g., those over the age of 65). In a preferred embodiment, the subject may be a human.

“Overweight” and “obesity” are both labels for ranges of weight that are greater than what is generally considered healthy for a given height. The terms also identify ranges of weight that have been shown to increase the likelihood of certain diseases and other health problems. An adult who has a BMI of between 25 and 25.9 is generally considered overweight. An adult who has a BMI of 30 or higher is generally considered obese. However, anyone who needs or wishes to reduce body weight or prevent body weight gain can be considered to be overweight or obese. Morbid obesity typically refers to a state in which the BMI is 40 or greater. In embodiments of the methods described herein, subjects have a BMI of less than about 40. In embodiments of the methods described herein, subjects have a BMI of less than about 35. In embodiments of the methods described herein, subjects have a BMI of less than about 35 but greater than about 30. In other embodiments, subjects have a BMI of less than about 30 but greater than about 27. In other embodiments, subjects have a BMI of less than about 27 but greater than about 25. In embodiments, the subject may be suffering from or be susceptible to a condition associated with eating such as binge eating or food cravings.

In certain embodiments, methods are provided for increasing the circulating concentrations of one or more of GLP-1 (total), GLP-1 (active), GLP-2, GIP, oxyntomodulin, PYY (total), PYY3-36, CCK, glycentin, amylin, uroguanylin, insulin and C-peptide is increased by about 0.5% to about 1000% by administering a nutritive taste receptor agonist formulated for delayed release to enteroendocrine cells combined with and preferably within a food product for oral ingestion compared to placebo-controlled circulating concentration. In certain embodiments, the circulating concentration of one or more of GLP-1 (total), GLP-1 (active), GLP-2, GIP, oxyntomodulin, PYY (total), PYY3-36, CCK, glycentin, amylin, uroguanylin, insulin and C-peptide is increased by about 0.5% to about 500% compared to placebo-controlled circulating concentrations. In certain embodiments, the circulating concentration of one or more of GLP-1 (total), GLP-1 (active), GLP-2, GIP, oxyntomodulin, PYY (total), PYY3-36, CCK, glycentin, amylin, uroguanylin, insulin and C-peptide is increased by about 0.5% to about 250% compared to placebo-controlled circulating hormone concentration. In certain embodiments, the circulating concentration of one or more of GLP-1 (total), GLP-1 (active), GLP-2, GIP, oxyntomodulin, PYY (total), PYY3-36, CCK, glycentin, amylin, uroguanylin, insulin and C-peptide is increased by about 0.5% to about 100% compared to placebo-controlled circulating concentration. In certain embodiments, the circulating concentration of one or more of GLP-1 (total), GLP-1 (active), GLP-2, GIP, oxyntomodulin, PYY (total), PYY3-36, CCK, glycentin, amylin, uroguanylin, insulin and C-peptide is increased by about 0.5% to about 75% compared to placebo-controlled circulating concentration. In certain embodiments, the circulating concentration of one or more of GLP-1 (total), GLP-1 (active), GLP-2, GIP, oxyntomodulin, PYY (total), PYY3-36, CCK, glycentin, amylin, uroguanylin, insulin and C-peptide is increased by about 0.5% to about 50% compared to placebo-controlled circulating concentration. In certain embodiments, the circulating concentration of one or more of GLP-1 (total), GLP-1 (active), GLP-2, GIP, oxyntomodulin, PYY (total), PYY3-36, CCK, glycentin, amylin, uroguanylin, insulin and C-peptide is increased by about 0.5% to about 35% compared to placebo-controlled circulating concentration. In certain embodiments, the circulating concentration of one or more of GLP-1 (total), GLP-1 (active), oxyntomodulin, PYY (total), PYY3-36, CCK, GIP, GLP-2, glycentin, uroguanylin, insulin, C-peptide and amylin is increased compared to placebo-controlled circulating concentration.

### Hormone Assays

In embodiments, the levels of hormones assayed in association with the methods of the invention, including, but not limited to, GLP-1, GLP-2, GIP, oxyntomodulin, PYY, CCK, glycentin, insulin, glucagon, ghrelin, amylin, uroguanylin, C-peptide and/or combinations thereof are detected according to standard methods described in the literature. For example, proteins can be measured by immunological assays, and transcription products by nucleic acid amplification techniques. Functional assays described in the art can also be used as appropriate. In embodiments, samples assayed comprise cultured cells, patient cell or tissue samples, patient body fluids, e.g., blood or plasma, etc. Similarly, the levels of analytes (e.g., glucose, triglycerides, HDL, LDL, apoB and the like) assayed in association with the methods of the invention are detected according to any known method.

For example, immunofluorescence can be used to assay for GLP-1. Cells can be grown on matrigel-coated cover slips to confluent monolayers in 12-well plates at 37° C., fixed in 4% paraformaldehyde in phosphate-buffered saline (PBS) and incubated with primary antiserum (e.g., rabbit anti-alpha gustducin, 1:150; Santa Cruz Biotechnology, and rabbit anti-GLP-1, Phoenix) overnight at 4° C. following permeabilization with 0.4% Triton-X in PBS for 10 minutes and blocking for 1 hour at room temperature. Following three washing steps with blocking buffer, the appropriate secondary antibody is applied (AlexaFluor 488 anti-rabbit immunoglobulin, 1:1000; Molecular Probes) for 1 hour at room temperature. After three washing steps, the cells can be fixed in Vectashield medium and the immunofluorescence visualized.

GLP-1 RNA isolated from cells can be assayed using RT-PCR. RT-PCR RNA isolation from cells can be performed using standard methodology. The RT-PCR reaction can be performed in a volume of 50 in a Peltier thermal cycler (PTC-225 DNA Engine Tetrad Cycler; MJ Research), using published primer sequences (Integrated DNA Technologies). Reverse transcription can be performed at 50° C. for 30 minutes; after an initial activation step at 95° C. for 15 minutes. PCR can be performed by denaturing at 94° C. for 1 minute, annealing at 55° C. for 1 minute and extension at 72° C. for 1 minute for 40 cycles, followed by a final extension step at 72° C. for 10 minutes. Negative controls can be included as appropriate, for example, by substituting water for the omitted reverse transcriptase or template. The control can be RNA isolated from, e.g., rat lingual epithelium. PCR products can be separated in 2% agarose gel with ethidium bromide, and visualized under UV light.

Radioimmunoassay (RIA) for total GLP-1 in patient blood samples can be performed as described in the art, e.g., by Laferrere, et al., 2007, “Incretin Levels and Effect are Markedly Enhanced 1 Month after Roux-en-Y Gastric Bypass Surgery in Obese Patients with Type 2 Diabetes, Diabetes Care 30(7):1709-1716 (using commercially available materials obtained from Phoenix Pharmaceutical, Belmont, Calif.). The authors describe measuring the effect of GIP and GLP-1 on secretion of insulin by measuring the difference in insulin secretion (area under the curve, or AUC) in response to an oral glucose tolerance test and to an isoglycemic intravenous glucose test.

Measurement of plasma concentrations of GLP-1, GIP, glucagon, insulin, C peptide, pancreatic peptide, nonesterified fatty acids, glutamic acid decarboxylase antibodies, and islet antigen antibodies, is described, e.g., by Toft-Nielsen, et al., 2001, “Determinants of the Impaired Secretion of Glucagon-Like Peptide-1 in Type 2 Diabetic Patients,” J. Clin. End. Met. 86(8):3717-3723. The authors describe the use of radioimmunoassay for GLP-1 to measure plasma concentrations of amidated GLP-1-(7-36), using antibody code no. 89390. This assay measures the sum of GLP-1-(7-36) and its metabolite GLP-1-(9-36). The authors describe measurement of GIP using C-terminally directed antibody code no. R65 (RIA), that reacts 100% with a human GIP but not with 8-kDA GIP.

GLP-1 and PYY can be directly assayed in the supernatant from venous effluents as described by, e.g., Claustre, et al. (1999, “Stimulatory effect of β-adrenergic agonists on ileal L cell secretion and modulation by α-adrenergic activation, J. Endocrin. 162:271-8). (See also Plaisancie' et al., 1994, “Regulation of glucagon-like peptide-1-(7-36) amide secretion by intestinal neurotransmitters and hormones in the isolated vascularly perfused rat colon,” Endocrinology 135:2398-2403 and Plaisancie' et al., 1995, “Release of peptide YY by neurotransmitters and gut hormones in the isolated, vascularly perfused rat colon,” Scandinavian Journal of Gastroenterology 30:568-574.) In this method, the 199D anti-GLP-1 antibody is used at a 1:250 000 dilution. This antibody reacts 100% with GLP-1-(7-36) amide, 84% with GLP-1-(1-36) amide, and less than 0.1% with GLP-1-(1-37), GLP-1-(7-37), GLP-2, and glucagon. PYY is assayed with the A4D anti-porcine PYY antiserum at a 1:800 000 dilution.

Methods for assaying GLP-1 and GIP are also described elsewhere in the art, e.g., by Jang, et al., PNAS, 2007.

PYY can also be assayed in blood using a radioimmunoassay as described by, e.g., Weickert, et al., 2006, “Soy isoflavones increase preprandial peptide YY (PYY), but have no effect on ghrelin and body weight in healthy postmenopausal women” Journal of Negative Results in BioMedicine, 5:11. Blood is collected in ice-chilled EDTA tubes for the analysis of glucose, ghrelin, and PYY. Following centrifugation at 1600 g for 10 minutes at 4° C., aliquots were immediately frozen at −20° C. until assayed. All samples from individual subjects were measured in the same assay. The authors described measuring immunoreactive total ghrelin was measured by a commercially available radioimmunoassay (Phoenix Pharmaceuticals, Mountain View, Calif., USA). (See also Weickert, et al., 2006, “Cereal fiber improves whole-body insulin sensitivity in overweight and obese women,” Diabetes Care 29:775-780). Immunoreactive total human PYY is measured by a commercially available radioimmunoassay (LINCO Research, Missouri, USA), using ¹²⁵I-labeled bioactive PYY as tracer and a PYY antiserum to determine the level of active PYY by the double antibody/PEG technique. The PYY antibody is raised in guinea pigs and recognizes both the PYY 1-36 and PYY 3-36 (active) forms of human PYY.

SGLT-1, the intestinal sodium-dependent glucose transporter 1, is a protein involved in providing glucose to the body. It has been reported to be expressed in response to sugar in the lumen of the gut, through a pathway involving T1R3 (Margolskee, et al., 2007 “T1R3 and gustducin in gut sense sugars to regulate expression of Na+-glucose cotransporter 1,” Proc Natl Acad Sci USA 104, 15075-15080”). Expression of SGLT-1 can be detected as described, e.g., by Margolskee, et al., for example, using quantitative PCR and Western Blotting methods known in the art. Measurement of glucose transport has been described in the literature, e.g., by Dyer, et al., 1997, Gut 41:56-9 and Dyer, et al., 2003, Eur. J. Biochem 270:3377-88. Measurement of glucose transport in brush border membrane vesicles can be made, e.g., by initiating D-glucose uptake by the addition of 100 of incubation medium containing 100 mM NaSCN (or KSCN), 100 mM mannitol, 20 mM Hepes/Tris (pH 7.4), 0.1 mM MgSO4, 0.02% (wt/vol) NaN3, and 0.1 mM D-[U¹C]glucose to BBMV (100 g of protein). The reaction is stopped after 3 sec by addition of 1 ml of ice-cold stop buffer, containing 150 mM KSCN, 20 mM Hepes/Tris (pH 7.4), 0.1 mM MgSO4, 0.02% (wt/vol) NaN3, and 0.1 mM phlorizin. A 0.9-ml portion of the reaction mixture is removed and filtered under vacuum through a 0.22-μm pore cellulose acetate/nitrate filter (GSTF02500; Millipore, Bedford, Mass.). The filter is washed five times with 1 ml of stop buffer, and the radioactivity retained on the filter is measured by liquid scintillation counting.

### Evaluation of Treatment of Obesity and Eating Disorders

In treatment of obesity it is desired that weight and/or fat is reduced in a subject. By reducing weight it is meant that the subject loses a portion of his/her total body weight over the course of treatment (whether the course of treatment be days, weeks, months or years). Alternatively, reducing weight can be defined as a decrease in proportion of fat mass to lean mass (in other words, the subject has lost fat mass, but maintained or gained lean mass, without necessarily a corresponding loss in total body weight). An effective amount of a nutritive sweet receptor agonist administered in this embodiment is an amount effective to reduce a subject's body weight over the course of the treatment, or alternatively an amount effective to reduce the subject's percentage of fat mass over the course of the treatment. In certain embodiments, the subject's body weight is reduced, over the course of treatment, by at least about 1%, by at least about 5%, by at least about 10%, by at least about 15%, or by at least about 20%. Alternatively, the subject's percentage of fat mass is reduced, over the course of treatment, by at least 1%, at least 5%, at least 10%, at least 15%, at least 20%, or at least 25%.

In embodiments wherein methods of treating, reducing, or preventing food cravings in a subject are provided, food cravings can be measured by using a questionnaire, whether known in the art or created by the person studying the food cravings. Such a questionnaire would preferably rank the level of food cravings on a numerical scale, with the subject marking 0 if they have no food cravings, and marking (if on a scale of 1-10) 10 if the subject has severe food cravings. The questionnaire would preferably also include questions as to what types of food the subject is craving.

### EXAMPLES

### Example 1

Tastants and Nutrient Synergy

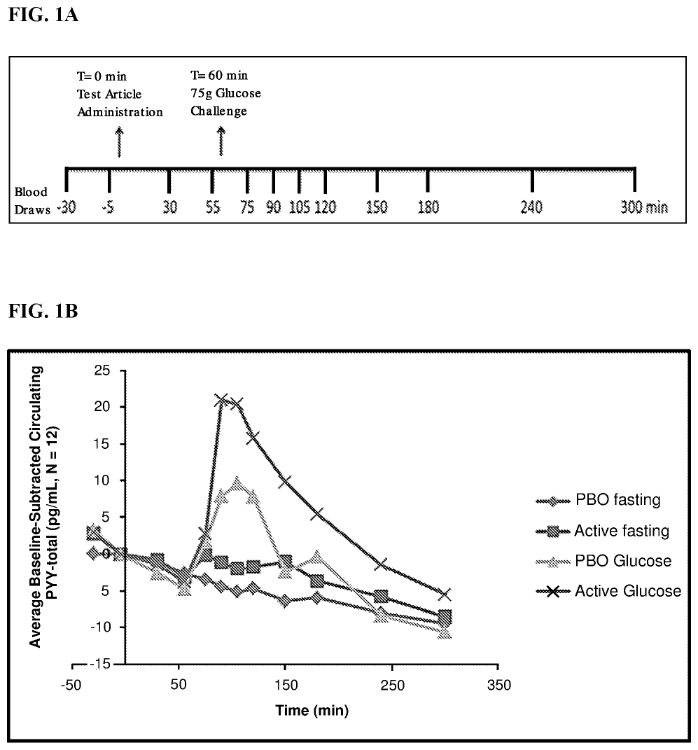

The effects of enteric coated tastants (Active) versus placebo (PBO) and nutrients (75 g glucose) on circulating gut hormone concentrations were evaluated in a 12 subject, randomized, double blind, placebo-controlled, crossover study that assessed the effects of tastants test article LC001 (pH sensitive enterically-coated tablets composed of Rebaudioside A, stevioside, sucralose, glutamine and quinine) and nutrients on the circulating gut satiety hormone PYY, using fully hydrogenated soybean oil as the coating. Subjects were administered the study test article in the morning after an overnight fast at t=0 min, and were given a 75 g glucose challenge at t=60 min, unless they were being tested for the fasting condition. Blood was sampled between t=−30 and 300 min relative to the dosing of test article for determination of gut hormone (PYY total) concentrations as depicted in FIG. 1A.

The results as illustrated in FIG. 1B of the experiment as described in paragraph [0082] are summarized here. Taste receptor agonists (tastants) synergistically promote elevation of circulating L-cell derived satiety hormone concentrations as exemplified by PYY total, i.e. nutrients amplify the effect of tastants.

### Example 2

A food bar of approximately 150 calories comprising 5 grams dates, 3 grams maple syrup, 3 grams almond butter, 5 grams almonds, 5 grams oats, 1 gram oat bran powder and 7 grams enterically-coated sugar particles has been tested for taste acceptability, ability to curb hunger, and suitability for use as an aid for body weight reduction.

### Example 3

Another food bar comprising 3 grams dates, 3.5 grams maple syrup, 3 grams almond butter, 3 grams peanut butter, 3 grams cashews, 3 grams almonds, 2.25 grams pumpkin seeds, 1 gram oat bran powder, and 7 grams enterically-coated sugar particles using fully-hydrogenated soybean oil as the coating was tested for taste acceptability, ability to curb hunger, and suitability for use as an aid for body weight reduction.

Eleven volunteers (7 females, 4 males, average age 60 y, 223.9 lbs) were instructed and trained to pursue an intermittent restricted daily calorie lifestyle by restricting daily caloric intake to no more than 500 Cals for 3 non-consecutive days each week. The volunteers attempted the lifestyle without the use of Bars for more than one week. After that, subjects were supplied with the Test Bars and asked to pursue the intermittent calorie restriction lifestyle for one week. Body weight was measured at the beginning and end of each week of calorie restriction. The volunteers were given a questionnaire for them to rate their Test Bars at the end of the study.

TABLE 1

| Ability of Bars to Curb Hunger | Response Rate |
|---|---|
| Not effective at all | 0% |
| Cannot tell whether it is effective | 0% |
| Somewhat effective | 3% |
| Effective | 70% |
| Very effective | 27% |

TABLE 2

| Usefulness of Bars for Alternate | |
|---|---|
| Day Calorie Restriction Lifestyle | Response Rate |
| Not useful | 0% |
| Cannot tell if it is useful | 0% |
| somewhat useful | 10% |
| Useful | 53% |
| Very useful | 37% |

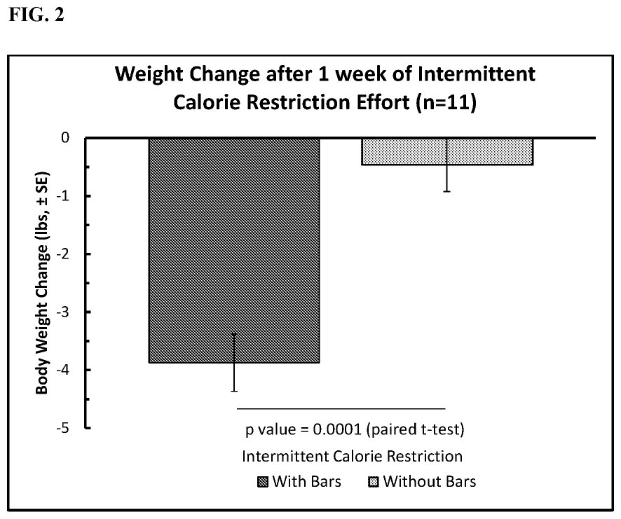

As shown in FIG. 2, the average weight loss for the individuals who consumed the food bars was significantly greater than the average weight loss for the individuals who did not consume the food bars.

### Example 4

In another food product such as the bars described in Examples 2-3, 0.3 gram of a coffee extract is added to the above composition to impart a coffee flavor to satisfy the coffee enthusiast.

### Example 5

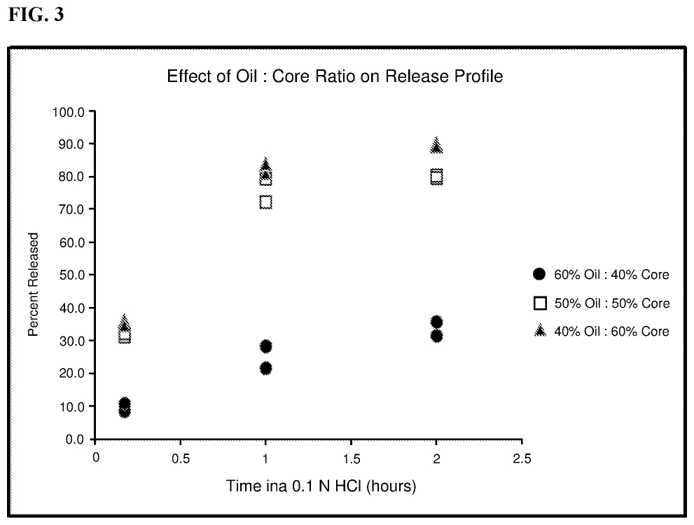

The effect of the composition of the coating on the release profile was evaluated in vitro. A nutritive taste receptor agonist was formulated for delayed release by employing a coating comprising fully hydrogenated soybean oil. In particular, compositions comprising 60% oil:40% core (w:w), 50% oil:50% core, or 40% oil:60% core were exposed to a medium of 0.1N HCl. The results as depicted in FIG. 3 show that the greater the percentage of oil, the longer the composition resists dissolution in an acidic environment such as the stomach.

### Example 6

A prospective randomized, age, breed, weight matched, placebo-controlled, crossover study is conducted evaluating the effectiveness of an embodiment of the presently claimed compositions to reduce daily kcal intake in dogs. A total of 20 dogs are evaluated in the 17 day study. The study design is as follows:

Treatment group: (N=10 dogs) Each dog receives a commercial formulation of the present invention (Lovidia™) 1 hour before their regularly scheduled meal each day. Total estimated kcal intake is calculated per dog per meal per day.
Placebo group: (N=10 dogs) Each dog receives a kcal matched treat 1 hour before their regularly scheduled meal each day. Total estimated kcal intake is calculated per dog per meal per day.
Crossover: After a 3 day wash out, no treatment or placebo, for both groups, the same 20 dogs are used. The two groups then crossover for an additional 7 days. The original treatment group receives the kcal matched treat and the previous placebo group receives Lovidia™.

1. A dietary composition comprising a bolus dose of a nutritive taste receptor agonist formulated for delayed release to enteroendocrine cells combined with a food product for oral ingestion.

2. The dietary composition according to claim 1, wherein said nutritive taste receptor agonist is a sweet receptor agonist.

3. The dietary composition according to claim 2, wherein said sweet receptor agonist is a sugar.

4. The dietary composition according to claim 3, wherein said sugar is glucose, fructose, or sucrose.

5. The dietary composition according to any one of claims 1 to 4, wherein said food product comprises a carbohydrate, a fat, a protein, or a combination thereof.

6. The dietary composition according to claim 5, wherein said food product comprises at least about 100 calories, at least about 150 calories, at least about 200 calories, at least about 250 calories, or at least about 300 calories.

7. The dietary composition according to 5, wherein said food product comprises less than about 500, less than about 450, less than about 400, or less than about 350 calories.

8. The dietary composition according to any one of claims 1 to 7, wherein said bolus dose is at least about 2 grams, at least about 3 grams, at least about 4 grams, at least about 5 grams, at least about 6 grams, at least about 7 grams, at least about 8 grams, at least about 9 grams, at least about 10 grams, at least about 12 grams, or at least about 15 grams of said taste receptor agonist.

9. The dietary composition of claim 1, wherein said nutritive taste receptor agonist is formulated to release in the duodenum, the jejunum, the ileum, the caecum, the colon, or combinations thereof.

10. The dietary composition according to any one of claims 1 to 9, wherein said taste receptor agonist is formulated with an enteric coating, a timed-release coating, or a combination thereof.

11. The dietary composition according to claim 10, wherein said timed-release coating comprises a hydrogenated fat, preferably a fully-hydrogenated fat.

12. The dietary composition according to claim 11, wherein said hydrogenated fat comprises at least about 50% (w/w) of said bolus dose.

13. The dietary composition according to claim 10, wherein said enteric coating is formulated to have an onset of release at a pH of about 5.0 or above, about 5.5 or above, about 6.0 or above, about 6.5 or above, about 7.0 or above, or combinations thereof.

14. The dietary composition according to any one of claims 1-13, wherein said composition comprises delayed-release particles of said taste receptor agonist incorporated into said food product.

15. The dietary composition according to claim 14, wherein said food product is provided in the form of a bar, cookie, cracker, wafer, or the like.

16. The dietary composition according to any one of claims 1-13, wherein said composition comprises a pre-measured bolus dose of delayed-release particles of said taste receptor agonist in a vial, cylinder, sachet or the like, for combination with a desired food product by a subject.

17. A method for controlling hunger, suppressing appetite, and/or inducing satiety in a subject comprising providing a composition according to any one of claims 1-16 to said subject.

18. The method according to claim 17, wherein less than about 25%, less than about 20%, less than about 15%, less than about 10%, less than about 5%, less than about 2.5%, or less than about 1% of said bolus dose is released in the stomach of said subject.

19. The method according to claim 17, wherein said administration increases the circulating concentration of GLP-1 (total), GLP-1 (active), GLP-2, PYY (total), PYY 3-36, or combinations thereof.

20. The method of claim 19, wherein the circulating concentrations of GLP-1 (active) and/or PYY (total) are increased by at least about 5%, by at least about 10%, by at least about 15%, by at least about 20%, by at least about 25%, or by at least about 50% compared to placebo-controlled circulating concentrations.

21. The method of any one of claims 17-20, wherein said subject is a human.

22. A kit comprising a bolus dose of microencapsulated particles comprising at least one nutritive taste receptor agonist formulated for delayed release to enteroendocrine cells, wherein said particles collectively comprise at least about 2 grams of said at least one taste receptor agonist, provided in a vial, cylinder, sachet or like container together with instructions for use in combination with food products.

23. The kit according to claim 22, wherein said at least one nutritive taste receptor agonist is a sweet receptor agonist; optionally wherein said sweet receptor agonist is a sugar a selected from the group consisting of glucose, fructose, or sucrose.

24. The kit according to claim 22 or 23, wherein said at least one taste receptor agonist is formulated with a timed-release coating comprising a hydrogenated fat, preferably a fully-hydrogenated fat.

25. The kit according to claim 24, wherein said hydrogenated fat comprises at least about 50% (w/w) of said bolus dose.
